(19) United States
(12) Patent Application Publication
(10) Pub. No.: US 2024/0298957 A1
(43) Pub. Date: Sep. 12, 2024

(54) SYSTEM AND METHOD FOR NEONATAL ELECTOPHYSIOLOGICAL SIGNAL ACQUISITION AND INTERPRETATION

(71) Applicant: UNIVERSITY COLLEGE CORK - NATIONAL UNIVERSITY OF IRELAND, CORK, Cork (IE)

(72) Inventors: Mark O'Sullivan, Cork (IE); Andriy Temko, Cork (IE); Alison O'Shea, Cork (IE); Geraldine Boylan, Cork (IE); Emanuel Popovici, Cork (IE)

(21) Appl. No.: 18/256,175

(22) PCT Filed: Dec. 7, 2021
(86) PCT No.: PCT/EP2021/084671

§ 371 Date: Jun. 6, 2023

(30) Foreign Application Priority Data
Dec. 7, 2020 (EP) 20212266.9

(51) Int. Cl. A61B 5/00; A61B 5/31; A61B 5/369

(52) U.S. Cl. CPC A61B 5/4094; A61B 5/0006; A61B 5/31; A61B 5/369; A61B 5/746; A61B 5/7405; A61B 5/742; A61B 2503/045

(57) ABSTRACT

The present invention relates to an integrated system and method for EEG signal acquisition and interpretation, for neonatal seizure detection. The system as per the present invention comprises a control device, an integrated circuit, a wireless communication module, a visual indication means, and a power management module. The control device has a Convolutional Neural Network (CNN) embedded on it, and is programmed to configure the integrated circuit to receive a plurality of channels of EEG data from a plurality of EEG acquisition electrodes, and to amplify and digitize the received EEG data. The integrated circuit is further configured to segment the channels of EEG data to a plurality of sequential epochs of EEG data. The control device is configured to pass the sequential epochs of EEG data through the CNN which is pre-trained to output the probability of a seizure in the EEG data passed through it. The channels of EEG data received from the acquisition electrodes and the probability of seizure outputted by the CNN is communicated to at least remote computing device by the communication module. A filter is applied to smooth the outputs in a post-processing routine. The visual indication means is enabled if the output of the CNN exceeds a predetermined threshold probability.

### FIELD

The present disclosure relates to a system and method for neonatal electrophysiological signal acquisition and interpretation.

### BACKGROUND

It is estimated that around four hundred thousand babies are born globally every day. Out of this approximately fifty thousand new-born babies are admitted to neonatal intensive care, and over two thousand new-born babies sustain brain injury every day.

The overall incidence rate of neonatal brain injuries occurring at, or soon after birth is approximately five per one thousand births (5 per 1,000) in high income countries. The incidence rate in premature births is approximately twenty six per one thousand (26 per 1,000). The incidence rates in low to middle income countries is significantly higher. Neonatal brain injuries include, but are not limited to, seizures, hypoxic-ischemic encephalopathy, stroke, intracranial haemorrhage, central nervous system infection and kernicterus and preterm infants with cystic periventricular leucomalacia. It results in the death or disability, such as epilepsy, cerebral palsy and cognitive impairment, of over one million infants globally each year, making it the fifth leading cause of death in children under five.

Seizures can be present across multiple types of neonatal brain injuries and are generally one of the most common manifestations and diagnostic indications of neonatal brain injuries. Seizures are notoriously difficult to diagnose since only 34% of seizures show clinical signs. Diagnosing seizures in neonates is further difficult as they do not always exhibit obvious behavioural change during a seizure.

It is estimated that around four hundred thousand babies are born globally every day. Out of this approximately fifty thousand new-born babies are admitted to neonatal intensive care, and over two thousand new-born babies sustain brain injury every day.

The overall incidence rate of neonatal brain injuries occurring at, or soon after birth is approximately five per one thousand births (5 per 1,000) in high income countries. The incidence rate in premature births is approximately twenty six per one thousand (26 per 1,000). The incidence rates in low to middle income countries is significantly higher. Neonatal brain injuries include, but are not limited to, seizures, hypoxic-ischemic encephalopathy, stroke, intracranial haemorrhage, central nervous system infection and kernicterus and preterm infants with cystic periventricular leucomalacia. It results in the death or disability, such as epilepsy, cerebral palsy and cognitive impairment, of over one million infants globally each year, making it the fifth leading cause of death in children under five.

Seizures can be present across multiple types of neonatal brain injuries and are generally one of the most common manifestations and diagnostic indications of neonatal brain injuries. Seizures are notoriously difficult to diagnose since only 34% of seizures show clinical signs. Diagnosing seizures in neonates is further difficult as they do not always exhibit obvious behavioural change during a seizure.

Electroencephalography (EEG) is the gold standard for monitoring brain function and diagnosing abnormal function such as HIE and seizures. Without EEG support, medical staff can only correctly diagnose nine percent (9%) of neonatal seizures. However, conventional EEG monitors are complex, heavy-duty systems which need to be rolled into the ward and take up to one hour to configure.

Conventional EEG monitors are also quite expensive and require multiple components such as a head-box/amplifier-box, laptop/computer, monitor screen and mains power connection. All these components require wired connections, in addition to multiple electrode leads that must be attached to the patient. Conventional EEG monitors are complex systems that consists of individual sub-systems that are mounted on a trolley, including an amplifier box, computing device and monitoring screen. Each sub system generally requires mains power. Each of the sub-systems require manual configuration and set up, including configuring the electrode inputs to the amplifier box, configuring the channel montage from the amplifier box inputs, configuring the data pre-processing on the computing device, and configuring the display of the continuous EEG traces on the monitoring screen for interpretation. These tasks require significant effort and time, and can cause significant disruption and stress to the infant. Furthermore, they rely on specialized medical staff with neurophysiology expertise to configure the equipment and interpret the resulting EEG, which inhibits its use in up to 80% of hospitals. A neurophysiologist is required to review hours of EEG data to identify abnormal activity. This equipment and expertise are also generally limited to tertiary-care hospitals with neurophysiology facilities. Even in such tertiary-care hospitals, the process of monitoring/diagnosing new-born babies suffers from long delays, making it difficult to treat within the optimal therapeutic window.

Prior art patent WO2010115939 presents a method for real-time identification of seizures in an EEG signal using a multi-patient trained generic Support Vector Machine Classifier which can be used to diagnose seizures in all patient types without the presence of a clinician. Further prior art patent CN 105395193A discloses an EEG acquisition device which enables analysis and interpretation of EEG data by transmitting the EEG signals to one or more computing devices.

Other examples of prior art systems include US2020/188697; WO2020/006263 and U.S. Pat. No. 10,743,809. US2020/188697 describes a system for stimulation and monitoring includes four individual and separate devices/systems, namely a brain monitor and stimulation wearable device; a mobile device, and a cloud, and a web app. WO2020/006263 describes a method for training data and training a machine learning model based on the acquired and subsequently tagged data. U.S. Pat. No. 10,743,809 describes a system for seizure prediction and detection uses traditional machine learning methods to predict seizure burden. The system described uses a feature extraction routine which is not accurate.

None of the prior art methods and processes, disclose a user-friendly means for EEG monitoring and processing with real-time decision support for neonatal seizure diagnosis accurately using a single device without requiring the support of an external computing device. Similarly, none of the prior art systems present an end-to-end system, operable by medical staff without neurophysiology expertise/training.

There is therefore an unfulfilled and unresolved need in the art for an easy to use and cost effective system and method which acquires and processes EEG data and enables real time decision support for neonatal seizure detection without reliance on highly skilled clinicians and complex external computing devices.

### SUMMARY

The present invention relates to an integrated system and method for neonatal EEG signal monitoring and interpretation and enables real time decision support for detecting neonatal seizures using a convolutional neural network, as set out in the appended claims.

In a preferred embodiment of the present invention, there is provided an integrated system for neonatal EEG acquisition and interpretation. The integrated system comprises a single module that contains a micro-controller unit, an analog front-end integrated circuit, a wireless communication integrated circuit, a means of providing a visual or auditive alert, and a power management module. The control unit has embedded on it, a trained convolutional neural network which enables classification of raw EEG data without a feature extraction stage. The control unit control device is operably interfaced to the analog front-end integrated circuit, communication module and the visual indication means. In an embodiment of the present invention, the control unit is operably interfaced to the analog front-end integrated circuit through a Serial Peripheral Interface (SPI). The communication module is operably interfaced to the control unit. The control unit, the analog front-end integrated circuit, the communication module, the power management module, and the visual indication means, are integrated to a single printed circuit board.

In an embodiment of the present invention, the integrated printed circuit board is enclosed in a casing which is approximately fifty centimetre cube (50 cm³) in size.

The analog front-end integrated circuit is configured to receive to receive a plurality of channels of EEG data from a plurality of EEG acquisition electrodes, and is configured to amplify and digitize the received plurality of channels of EEG data. In an embodiment of the present invention, the plurality of channels of EEG data comprises eight (8) channels of EEG data. The analog front-end integrated circuit is also configured to transmit the EEG data from the plurality of EEG channels to the control unit. In an embodiment of the present invention, the integrated circuit is an eight (8) channel, twenty four (24) bit programmable gain amplifier and analog to digital converter.

The control unit is configured to receive the EEG data from the plurality of EEG channels, to filter and down sample the EEG data and to segment said data into a plurality of sequential epochs of EEG data. In an embodiment of the present invention, the plurality of sequential epochs of EEG data comprises of eight (8) second epochs with a fifty percent (50%) overlap between successive windows. The sequential epochs of EEG data are subsequently used as input to the convolutional neural network. In an embodiment of the present invention the control unit is a low power microcontroller, for example STF32F401.

The convolutional neural network is adapted to output the probability of occurrence of a seizure in the inputted plurality of sequential epochs of EEG data. A post-processing stage is included to smooth the outputs using a moving average filter to reduce the number of false alarms. In an embodiment of the present invention, a convolutional neural network with ten convolutional layers and no fully connected layer used.

It will be appreciated that the system can be pre-configured without requiring user input, whereas all existing systems require a laptop/tablet/PC to configure the analog front-end's channels, filtering, sampling rate, amplification, and impedance checking. The present invention allows for operations to be hard-coded on the control unit and transmitted to the analog front-end integrated circuit upon startup, meaning after startup, the device is configured and ready to record EEG using the standard configuration without any further configuration requirements.

The control unit is further configured to provide an alarm or alert, by visual or auditive means, if the output of the convolutional neural network exceeds a predetermined threshold probability value. In an embodiment of the present invention, said predetermined threshold probability value is fifty percent (50%) probability of the occurrence of a seizure.

The control unit is interfaced with the communication integrated circuit to communicate in real time the plurality of channels of EEG data and the output of the convolutional neural network, to a server or device for data storage. The communication module could be for example, a Bluetooth module or a Wireless Fidelity module or any other wireless communication means.

In a preferred embodiment of the present invention, a method for neonatal Electroencephalogram (EEG) acquisition and interpretation, is provided. The method comprises the steps of firstly receiving a plurality of channels of EEG data from a plurality of EEG acquisition electrodes. The received plurality of channels of EEG data is amplified and digitized, and transmitted to a control unit. The data is filtered and down sampled on the control unit and segmented into a plurality of sequential epochs of EEG data. The control unit has a convolutional neural network embedded on it, to which the sequential epochs of EEG data is inputted. The convolutional neural network is adapted to output the probability of occurrence of a seizure in the inputted plurality of sequential epochs of EEG data. The output of the convolutional neural network and the plurality of channels of EEG data received from the EEG acquisition electrodes, are communicated in real time to a server or cloud platform or connected device. Further, if the output of the convolutional neural network exceeds a predetermined threshold probability value, for example fifty percent (50%), a visual or auditive means of alarm is triggered to notify a clinician or a care giver regarding a higher probability of occurrence of an event of neonatal seizure.

The present invention utilizes edge inference of machine learning to provide clinicians with real-time diagnostic decision support on a single device. Machine learning algorithms for detecting brain injury in new-born babies are being rapidly developed in today's world. The ability to seamlessly integrate such algorithms is a unique advantage of the present invention. The compact, wireless, standalone, and user-friendly design of the system comprising the present invention makes it readily available for use by clinicians with minimal delay and complexity. The present invention is a disruptive technology in its relevant domain as it would enable all new-born babies in high risk pregnancy to be easily and quickly screened after birth for potential brain injury.

The present invention resolves the deficiencies in the art by providing a point-of-care EEG monitoring solution that requires minimum set-up time and is usable by a much wider demographic of medical staff compared to existing solutions. The plug and play nature of the present invention, allows medical staff without any neurophysiological expertise to plug an off-the-shelf EEG headcap into the present invention. The standalone design of the system comprising the present invention provides all the necessary functionalities without complex machinery and operation and provides accurate decision support within seconds.

By using real-time machine learning algorithms capable of detecting abnormal EEG with great accuracy within the system, the present invention improves accuracy, usability, and timeliness for detecting neonatal seizures, in comparison to prior art systems and methods. Enabling quick and user-friendly acquisition of EEG with real-time diagnostic decision support is a disruptive breakthrough in the field of brain monitoring. Further, the neonatal brain injury detection algorithms embedded in the control unit are optimized for use in low-power applications, which allows them to be implemented locally in the present invention without the need for an external or remote computing device.

In addition to deployment for neonatal acute care, the present invention could be used in pediatric and adult ICU's and could be used to diagnose patients with for example, suspected non-conclusive status epilepticus.

In one embodiment the post-processing routine comprises a moving average filter.

In one embodiment the moving average filter includes a binarization step configured to smooth the output and improve the classification accuracy.

In one embodiment the number of channels is less than eight and wherein the epoch size is less than or equal to eight seconds.

In one embodiment the post-processing routine comprises a bandpass filtering and a down-sampling step. In one embodiment the convolutional neural network comprises a classification structure having non fully-connected layers.

In one embodiment the classification comprises one or more of the following neonatal seizure detection; neonatal neurological health; onset of abnormal neurological events.

It will be appreciated that the integrated system according to the invention can be placed inside the incubator without requiring external cables/wires and thus reducing the number of disturbances to the infant which promotes healthy growth. The system can be easily configured with an IoT mesh whereby be recording and transmitting data to a WiFi router (station) to a server for data storage and review.

As the data processing chain is completed locally on the micro-controller unit, there is minimal risk for electromagnetic interference, lost data packets, latency in data transfer, and failed transfer as is associated with wireless communication protocols.

The present invention hence provides a robust solution and optimal solution to problems identified in the art. Other advantages and additional novel features of the present invention will become apparent from the subsequent detailed description.

There is also provided a computer program comprising program instructions for causing a computer program to carry out the above method which may be embodied on a record medium, carrier signal or read-only memory.

### BRIEF DESCRIPTION OF DRAWINGS

The invention will be more clearly understood from the following description of an embodiment thereof, given by way of example only, with reference to the accompanying drawings, in which:—

FIG. 1 is a schematic diagram of a system illustrating a preferred embodiment of the present invention; and

FIG. 2 is a flow diagram illustrating a method as per a preferred embodiment of the present invention.

### DETAILED DESCRIPTION OF DRAWINGS

The present invention relates to a system and method for neonatal electrophysiological signal acquisition and interpretation, and more particularly to a system and method for EEG signal acquisition and interpretation for neonatal seizure detection.

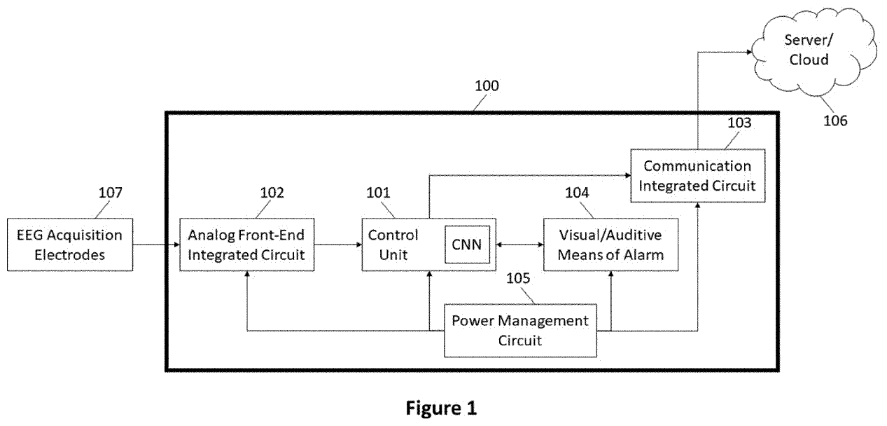

Referring to FIG. 1, the system 100 as per the present invention comprises a control unit 101, an analog front-end integrated circuit 102, a communication integrated circuit 103, a visual indication means 104, and a power management circuit 105. The control device 101 is operably interfaced to the integrated circuit 102 and the visual/auditive means of alarm 104. The communication integrated circuit 103 is operably interfaced to the control unit 101. In an embodiment of the present invention, the control unit 101, the analog front-end integrated circuit 102, the communication integrated circuit 103, the power management circuit 104, and the visual/auditive means of alarm 105, are integrated to a printed circuit board which in turn is enclosed in a casing which is approximately fifty centimetre cube (50 cm³) in volume.

In an embodiment of the present invention, the control unit 101 is a low power microcontroller, for example a STM32F401 microcontroller. The control unit 101 has embedded on it a convolutional neural network which is pre-trained for neonatal seizure detection. Convolutional neural networks can be configured to provide classification of raw EEG data without a feature extraction stage. Said convolutional neural network is deployed to the control unit 101 using a compatible expansion pack or other suitable library source that enables integration and optimization of neural networks for deployment in microcontrollers.

The control unit 101 is adapted to configure the analog front-end integrated circuit 102 to receive a plurality of channels of EEG data from a plurality of EEG acquisition electrodes 107. The plurality of EEG acquisition electrodes 107 can be positioned on an electrode cap (not shown) fitted to the head of an infant and configured to measure the neonatal signals and provide to the system 101 of the present invention. In an embodiment of the present invention the control unit 101 is operably interfaced to the analog front-end integrated circuit 102 through a Serial Peripheral Interface (SPI). The analog front-end integrated circuit 102 is configured to amplify and digitize the plurality of channels of EEG data received from the EEG acquisition electrodes. In an embodiment of the present invention the analog front-end integrated circuit 102 is an eight channel-twenty four bit-programmable gain amplifier and analog to digital converter having low power consumption, high resolution, high input impedance, and a small package footprint, such as the ADS1299 ASIC. The analog front-end integrated circuit 102 is also configured to filter and down sample the plurality of channels of EEG data. In an embodiment of the present invention, the plurality of channels of EEG data comprises eight (8) channels of EEG data from the plurality of EEG acquisition electrodes. The analog front-end integrated circuit 102 is further configured to transmit the EEG data from the plurality of EEG channels at a fixed sampling rate, for example two hundred and fifty (250) Hertz, to the control unit 101.

The control unit 101 is programmed to receive the EEG data, to filter and down sample the channels of EEG data, to split the EEG data into a plurality of sequential epochs of EEG data, to pass it through the convolutional neural network, to post-process the outputs of the convolutional neural network using a smoothing filter. The output of the convolutional neural network is the probability of occurrence of a seizure in the inputted plurality of sequential epochs of EEG data. In an embodiment of the present invention, the convolutional neural network comprises of 10 convolutional layers trained to output the probability of occurrence of an event of a seizure in the EEG window passed through it. The control unit 101 is configured to trigger a visual or auditive means of alarm 104, for example a flashing LED, if the output of the convolutional neural network exceeds a threshold probability percentage. In an embodiment of the present invention, said threshold probability percentage is fifty percentage (50%), and the means of alarm 104 is triggered to alert a clinician or a care giver of the new-born, if the output of the convolutional network exceeds 50%. This ensures that the clinician or care giver closest to the new-born baby is alerted instantaneously without requiring an external means such as a monitor screen. It will be appreciated that the probability threshold can be selected in a number of different ways and can be selected as a trade-off between the sensitivity and number of false detections. A simple selection for the CNN network is to chose halfway point, so greater than 50% probability is more likely to be a seizure than not. The threshold value may change depending on whether a user wants to reduce the number of false alarms while still detecting the majority of seizure burden, or else to have greater sensitivity at the expense of more false alarms. Suitably a traffic light system can be used of red, green and amber depending on the seriousness of the seizures detected.

The control unit 101 is further adapted to configure the communication integrated circuit 103 to communicate in real time the plurality of channels of EEG data and the output of the convolutional neural network, to a server or cloud platform 106. This enables retrospective review of the channels of EEG and outputs of the convolutional neural network remotely. The server or cloud platform 106 may be for example, a hospital server, a personal computer, a portable device such as a tablet computer, a laptop, a smart phone, a medical device, or any cloud server. The communication integrated circuit 103 could be for example, a Bluetooth module or a Wireless Fidelity module or any other wireless communication means which enables wireless communication to external computing devices.

The power management circuit 105 is operably coupled to the analog front-end integrated circuit 102, the control unit 101, and the communication integrated circuit 103. In an embodiment of the present invention, the power management circuit 105 comprises a 3.7V Lithium Polymer battery, and the system 100 is adapted to charge the battery via a micro-USB charging circuit, and to regulate voltage of each components in the circuit. While the system 100 is operational, the micro-USB charging circuit is disconnected, and while the system 100 is non-operational, the micro-USB charging circuit is connected. In said embodiment, there is therefore isolation between the patient and the charging circuit since the charging circuit may be connected to mains power. In said embodiment, the total current consumption of the system 100 was in the range of milliamperes and provides a battery-life of approximately 24 hours.

It will be appreciated that the invention provides a single and standalone, module system 100 that can be easily interfaced with an electrode headcap facilitates a system 100 that can be configured by non-experts in multiple clinical settings; unlike existing systems. The invention facilitates the full end-to-end EEG data processing chain on a single module without requiring user-input for acquisition or interpretation configuration. A standalone system that can be placed in an incubator with an infant without requiring user-input or protruding cables and leads presents a significant advance on the current clinical practice.

Convolutional neural networks (CNN) consist of several layers of interconnected weights and activations which typically culminate in a fully connected layer to classify the output of the CNN. Prior art CNNs, that achieve state-of-the-art accuracy, in the field can often contain hundreds of thousands of trainable parameters. Deployment of such CNNs require megabytes of available memory, which is not available in low-power micro-controller units.

The CNN algorithm for EEG classification and interpretation herein included on the single module has been specially adapted to deliver state-of-the-art results despite the constraints of the device's size. A family of algorithms utilise minimally pre-processed EEG; that are capable of detecting brain abnormalities in temporal EEG signals. This approach provides a novel movement away from algorithms which require EEG signals, or any physiological signal, to be decomposed into representative features; calculating features requires memory, compute power, and is time intensive, this makes them unsuitable for inclusion in a single module system. Circumventing the need for representative features in the algorithm allows for the processing of the signals included on the single module.

The cost of using minimally pre-processed temporal EEG data is that they are noisier and higher-dimensional than representative features, so the algorithms developed overcome this challenging task. The invention provides a CNN algorithm architecture which can handle the increased ambiguity in the input data required extensive experimentation and with the added constraint of developing an architecture which is light-weight. This is a necessary requirement in order for the algorithm to fit within the memory, computational, and temporal constraints of the single module system. In other words the invention provides an application-specific CNN algorithm to meet these challenges and is easily adapted to fit into the system.

This involved removing the most feature-heavy, fully-connected layers which are typically used to classify in CNN algorithms and replacing it with simple convolutional filters; this is the step which makes the algorithm ‘fully-convolutional’. Unlike prior art which uses lossy methods of compressing the size of neural networks at the cost of classification accuracy, the algorithm achieves state-of-the-art performance and light-weight architecture without compromising classification accuracy. Unlike typical CNNs with hundreds of thousands of parameters, the specially adapted CNN contains less than 26,000 parameters, making it feasible for low-power embedded systems deployment. In the context of the present invention EEG requires both high amplitude and temporal resolution. EEG is generally recorded at a minimum of 250 Hz (samples per second) with a resolution of 24 bits. Eight channels of EEG therefore produce 384,000 bits of data per second, and for context, sixty seconds results in 23,040,000 bits per second. In addition to the continuously acquired EEG data on the micro-controller unit, the convolutional neural network weights and activations require memory, storage, and computational overhead. These data rates and requirements present a significant challenge for real-time data processing and inference of automated classification models. The system of the present invention deploys hardware, firmware, and deep learning architecture design levels to facilitate the end-to-end data chain on a single low-power device without compromising the clinical accuracy of the diagnostic decision support.

As a specific non limiting example, data is down sampled to 50 Hz, reducing the data-rate by a factor of five. To avoid aliasing, this requires implementation of a low-pass filter with a cut-off frequency set at least below half of the sampling frequency (25 Hz). For the task of neonatal seizure detection, clinical review of EEG has shown that there is minimal pertinent information in EEG signals in frequencies above 25 Hz. The prominent frequencies observed in neonatal seizures vary from 0.5 Hz to 4 Hz. Therefore, a window length of at least two seconds is required to capture a single repetition of the prominent frequency. Using eight second epochs provides a sufficiently-sized window to observe the evolution of the temporal and frequency content of the EEG signal and allow a deep convolutional neural network to learn the features of seizure activity in an EEG epoch. However, it is sufficiently short to provide probabilistic output on the acquired EEG at near real-time without significant latency, while also reducing the amount of data to be stored on the micro-controller unit at any given time.

It will be further appreciated that in a preferred embodiment of the present invention, the full data chain is managed and completed on a single micro-controller unit, including data acquisition, data segmentation, data pre-processing, inference of convolutional neural network models, post-processing of convolutional neural network model outputs, and means of providing continuous diagnostic indications to the user.

The data transmitted from the analog front-end integrated circuit over serial peripheral interface to the micro-controller unit is received in a known channel configuration. The conversion of the referential channel inputs to a bi-polar montage on the micro-controller unit facilitates timely and user-agnostic configuration. Segmentation and pre-processing of the received EEG data on the micro-controller unit enables deployment and inference of convolutional neural network model at device level. Storage of the convolutional neural network weights and activation on the micro-controller unit facilitates on-board computation of the probabilistic value of a given EEG epoch containing seizure activity. Post-postprocessing of the probabilistic outputs of the convolutional neural network model on the micro-controller unit smooths the probabilistic outputs which reduces variability of the output to provide a smoothed output, and thus, results in a significantly lower rate of false detections.

In a preferred embodiment of the present invention, a system for neonatal EEG acquisition and interpretation is provided. The system comprises a single module that embeds an analog-front end (AFE) integrated circuit (IC), a micro-controller unit (MCU), a communication module, a means of providing a diagnostic output indication, and a power management module. The micro-controller unit has embedded thereon along with a trained convolutional neural network which enables classification of raw EEG data without a feature extraction stage. The micro-controller unit is operably interfaced to the analog front-end integrated circuit, communication module and the visual indication means. In an embodiment of the present invention, the micro-controller device is operably interfaced to the analog front-end integrated circuit through a Serial Peripheral Interface (SPI). The communication module is operably interfaced to the micro-controller control device. The control device, the integrated circuit, the communication module, the power management module, and the visual indication means, are integrated to a single printed circuit board.

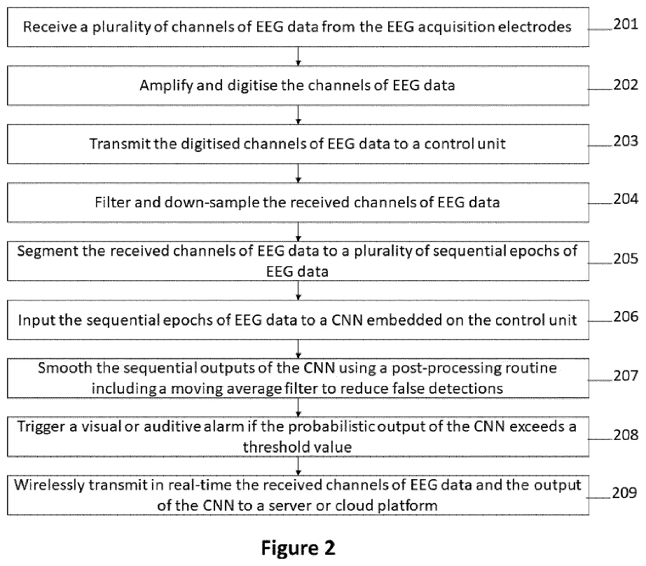

FIG. 2 illustrates a method as per a preferred embodiment of the present invention. The method comprises the steps of receiving a plurality of channels of EEG data from a plurality of EEG acquisition electrodes 201. The received plurality of channels of EEG data is amplified and digitized 202 and transmitted to a control unit 203. The received channels of EEG data is filtered and down sampled on the control unit 204. In an embodiment of the present invention, the plurality of channels of EEG data comprises eight (8) channels of EEG data.

In an embodiment of the present invention, the EEG data from a plurality of EEG channels is split into sequential epochs of EEG data comprising of eight (8) seconds on the control unit 205. The sequential epochs are inputted to a pre-trained convolutional neural network embedded in the control unit 206. The probabilistic outputs of the convolutional neural network are smoothed using a moving average filter on the control unit 207. The convolutional neural network is adapted to output the probability of occurrence of a seizure in the inputted plurality of sequential epochs of EEG data. A visual or auditive means of alarm is triggered if the output of the convolutional neural network exceeds a threshold probability percentage 208. In an embodiment of the present invention, said threshold probability percentage is fifty percent (50%). The plurality of channels of EEG data received from the acquisition electrodes and the output of the convolutional neural network is communicated to a server or cloud platform 209. This enables retrospective review of the channels of EEG and outputs of the convolutional neural network remotely.

Although the present invention has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present invention as defined.

Further, a person ordinarily skilled in the art will appreciate that the various illustrative method steps described in connection with the embodiments disclosed herein may be implemented using electronic hardware, or a combination of hardware and software. To clearly illustrate this interchangeability of hardware and a combination of hardware and software, various illustrations and steps have been described above, generally in terms of their functionality. Whether such functionality is implemented as hardware or a combination of hardware and software depends upon the design choice of a person ordinarily skilled in the art. Such skilled artisans may implement the described functionality in varying ways for each particular application, but such obvious design choices should not be interpreted as causing a departure from the scope of the present invention.

The method described in the present disclosure may be implemented using various means. For example, the system described in the present disclosure may be implemented in hardware, firmware, software, or any combination thereof. For a hardware implementation, the processing units, or processors(s) or controller(s) may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

For a firmware and/or software implementation, software code may be stored in the memory means and executed by a processor. The memory means may be implemented within the processor unit or external to the processor unit. As used herein the term “memory” refers to any type of volatile memory or non-volatile memory.

The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. a memory stick or hard disk.

The carrier may be an electrical or optical signal which may be transmitted via an electrical or an optical cable or by radio or other means. In the specification the terms “comprise, comprises, comprised and comprising” or any variation thereof and the terms include, includes, included and including” or any variation thereof are considered to be totally interchangeable and they should all be afforded the widest possible interpretation and vice versa.

The invention is not limited to the embodiments hereinbefore described but may be varied in both construction and detail.

1. An integrated system for neonatal Electroencephalogram (EEG) acquisition and interpretation, the system comprising:
a control unit having embedded on it a convolutional neural network, the control unit operably interfaced to an analog front-end integrated circuit and a visual or auditive means of alarm; and
a communication integrated circuit operably interfaced to the control unit; and
characterised in that the control unit is adapted to:
configure the analog front-end integrated circuit to receive a plurality of channels of EEG data from a plurality of EEG acquisition electrodes, amplify and digitize the received plurality of channels of EEG data, and transmit the EEG data from the plurality of EEG channels to the control unit;
read the EEG data from the plurality of EEG channels transmitted from the analog front-end integrated circuit;
filter and down sample the EEG data in a pre-processing routine;
segment the plurality of channels of EEG data into a plurality of sequential epochs of EEG data;
input the plurality of sequential epochs of EEG data to the convolutional neural network, the convolutional neural network is adapted to output the probability of occurrence of a seizure in the inputted plurality of sequential epochs of EEG data;
apply a filter to smooth the outputs in a post-processing routine;
configure the communication integrated circuit to communicate in real time the plurality of channels of EEG data and the output of the convolutional neural network, to a server or cloud based platform; and
trigger the visual or auditive means of alarm if the output of the convolutional neural network exceeds a predetermined threshold probability value.

2. The integrated system as claimed in claim 1 wherein the analog front-end integrated circuit is an eight channel twenty four bit programmable gain amplifier and analog to digital converter.

3. The integrated system as claimed in any of the preceding claims, wherein the plurality of channels of EEG data comprises eight channels of EEG data.

4. The integrated system as claimed in any of the preceding claims, wherein the plurality of sequential epochs of EEG data comprises eight second epochs with a fifty percent (50%) overlap between successive windows.

5. The integrated system as claimed in any of the preceding claims, wherein the control unit is operably interfaced to the integrated circuit through a Serial Peripheral Interface.

6. The integrated system as claimed in claim 1, wherein the communication module is a Bluetooth module.

7. The integrated system as claimed in claim 1, wherein the communication module is a Wireless Fidelity module.

8. The integrated system as claimed in any of the preceding claims, further comprising a power management circuit.

9. The integrated system as claimed in claim 8, wherein the power management circuit includes a lithium polymer battery.

10. The integrated system as claimed in any of the preceding claims, wherein the predetermined threshold probability value is fifty percent (50%).

11. The integrated system as claimed in any of the preceding claims, wherein the control unit, the analog front-end integrated circuit, communication integrated circuit, the power management circuit, and the visual or auditive means of alarm, are integrated to a printed circuit board.

12. The integrated system as claimed in any preceding claim wherein the post-processing routine is a moving average filter.

13. The integrated system as claimed in claim 12 wherein the moving average filter includes a binarization step configured to smooth the output and improve the classification accuracy.

14. The integrated system as claimed in any preceding claim wherein the number of channels is less than eight and wherein the epoch size is less than or equal to eight seconds.

15. The integrated system as claimed in any preceding claim wherein the post-processing routine comprises a bandpass filtering and a down-sampling step.

16. The integrated system as claimed in any preceding claim wherein the convolutional neural network comprises a classification structure having non connected layers.

17. The integrated system as claimed in claim 15 wherein the classification comprises one or more of the following neonatal seizure detection; neonatal neurological health; onset of abnormal neurological events

18. A method for neonatal Electroencephalogram (EEG) acquisition and interpretation, the method comprising the steps of:
a) receiving a plurality of channels of EEG data from a plurality of EEG acquisition electrodes;
b) amplifying and digitizing the received plurality of channels of EEG data;
c) transmitting the EEG data from a plurality of EEG channels to a control unit;
d) segmenting the received plurality of channels of EEG data into a plurality of sequential epochs of EEG data;
e) inputting the plurality of sequential epochs of EEG data to a convolutional neural network embedded in the control unit;
wherein the convolutional neural network is adapted to output the probability of occurrence of a seizure in the inputted plurality of sequential epochs of EEG data;
f) communicating in real time the plurality of channels of EEG data received in step (a) and the output of the convolutional neural network, to a server or cloud platform; and
g) trigger a visual or auditive means of alarm if the output of the convolutional neural network exceeds a predetermined threshold probability value.

19. The method as claimed in claim 18, further comprising the steps of filtering and down sampling the plurality of channels of EEG data.

20. The method as claimed in any of the preceding claims, wherein the plurality of channels of EEG data comprises eight channels of EEG data.

21. The method as claimed in any of the preceding claims, wherein the plurality of sequential epochs of EEG data comprises eight second epochs with a fifty percent (50%) overlap between successive windows.

22. The method as claimed in any of the preceding claims, wherein the predetermined threshold probability value is fifty percent (50%).
